SEQUENCE LISTING (5 sequences)

SEQ ID NO: 1 - DNA, 24 bp, Artificial Sequence
  Synthetic Oligonucleotide
cttgaatactacagcatggaataa

SEQ ID NO: 2 - DNA, 52 bp, Artificial Sequence
  Synthetic Oligonucleotide
  promoter (1)..(27)
aatttaatacgactcactatagggagattcacctctgacagttaaatacgaa

SEQ ID NO: 3 - DNA, 23 bp, Artificial Sequence
  Synthetic Oligonucleotide
caguccauaaugaaguaggacug

SEQ ID NO: 4 - DNA, 54 bp, Artificial Sequence
  Synthetic Oligonucleotide
  Capture Probe Second Segment (22)..(54)
uaggccaauacccuaccgucctttaaaaaaaaaaaaaaaaaaaaaaaaaaaaaa

SEQ ID NO: 5 - RNA, 1205 nt, Babesia microti
gggcgaauuggguaccgggcccccccucgaggucgacgcuuaguauaagcuuuuauacagcgaaacugcgaauggcucauuaaaacaguuauaguuuauuugauguucguuuuacauggauaaccgugguaauucuagggcuaauacaugcucgaggcgcguuuucgcguggcguuuauuagacuuuaaccaacccuucggguaaucggugauucauaauaaauuagcgaaucgcauggcuuugccggcgauguaucauucaaguuucugaccuaucagcuuuggacgguaggguauuggccaccggggcgacgacgggugacggggaauugggguucgauuccggagagggagccugagaaacggcuaccacaucuaaggaaggcagcaggcgcgcaaauuacccaauccugacacagggagguagugacaagaaauaacaauacagggcuuaaagucuuguaauuggaaugaugggaaucuaaacccuucccagaguaucaauuggagggcaagucuggugccagcagccgcgguaauuccagcuccaauagcguauauuaaaguuguugcaguuaagaagcucguaguugaauuucugccuugucauuaaucucgcuuccgagcguuuuuuuauugacuuggcaucuucuggauuuggugccuucggguacuauuuuccaggauuuacuuugagaaaacuagaguguuucaaacaggcauucgccuugaauacuacagcauggaauaaugaaguaggacuuugguucuauuuuguugguuauugagccagaguaaugguuaauaggagcaguugggggcauucguauuuaacugucagaggugaaauucuuagauuuguuaaagacgaacuacugcgaaagcauuugccaaggauguuuucauuaaucaagaacgaaaguuaggggaucgaagacgaucagauaccgucguaguccuaaccauaaacuaugccgacuagagauuggaggucgucaguuuaaacgacuccuucagcaccuugagagaaaucaaagucuuuggguucuggggggaguauggucgcaagucugaaacuuaaaggaauugacggaagggcaccaccaggcguggagccugcggcuuaauuugacucaacacgggaaaccucaccagguccagacauagagaggauugacagauugauagcucuuucuugaugaauu

(19) United States
(12) Patent Application Publication
(10) Pub. No.: US 2018/0143115 A1
(43) Pub. Date: May 24, 2018

(54) METHOD OF POOLING BLOOD SAMPLES

(71) Applicant: GEN-PROBE INCORPORATED, San Diego, CA (US)

(72) Inventors: Jeffrey M. LINNEN, San Diego, CA (US); Jijumon CHELLISERRY, San Diego, CA (US)

(21) Appl. No.: 15/573,014

(22) PCT Filed: May 12, 2016
(86) PCT No.: PCT/US2016/032011

§ 371 Date: Nov. 9, 2017

Related U.S. Application Data
(60) Provisional application No. 62/160,591, filed on May 12, 2015.

(51) Int. Cl. G01N 1/38; C12Q 1/6893; C12Q 1/6806; C12Q 1/70

(52) U.S. Cl. CPC G01N 1/38; C12Q 1/70; C12Q 1/6806; C12Q 1/6893

(57) ABSTRACT

The disclosure provides methods of pooled analysis of samples containing red blood cells, such as whole blood cell samples. The methods are particularly useful for screening whole blood samples collected from many individuals to be used for transfusions and the like. Aliquots of such samples are individually contacted with a lysis reagent, further described below, before pooling to lyse red blood cells in the samples. After pooling, the lysates, the combined lysate is tested for presence of target(s) characteristic of pathogen(s). The method are particularly useful for identifying pathogens of red blood cells, but can also be used to nm a complete panel of testing including for all of the typical targets.

### CROSS REFERENCE TO RELATED APPLICATIONS

This application claims the benefit of priority of U.S. provisional application No. 62/160,591, which was filed on May 12, 2015, and which is incorporated herein by reference.

### BACKGROUND

Blood transfusion has an essential role in patient management. Blood used for transfusion is often collected from many volunteers. Such samples are screened for transmissible infections, such as HIV-1, HIV-2, hepatatis B virus, hepatitis C virus, dengue virus, west nile virus, treponema pallidum, Anaplasma phagocytophilum, and depending on local prevalence, Trypanosoma cruzi and Plasmodium species among others.

Screening for additional pathogens would be desirable. For example, human Babesiosis is an emerging infectious disease which is a tick-borne intraerythrocytic infection. Babesia microti, the most common cause for human Babesiosis in the U.S is widely endemic in the Northeastern and upper Midwestern United States. This species has been implicated in majority of the transfusion-transmitted babesiosis (TTB) cases in the United States and is currently the most reported transfusion transmitted disease to the FDA. 159 well-documented cases of B. microti TTB were reported between 1979 and 2009 in the US (Herwaldt et al., Ann Intern Med 2011). 87% of transmitted infusions occurred in seven endemic US states. Twelve transfusion-associated fatalities associated with TTB have occurred since 2005.

Because the proportion of infected samples is low, screening can be simplified by pooling samples. If a pool sample is infected, the pool can be deconvoluted to identify the source of infection in an individual sample, but if the pool is not infected, all samples contributing to the pool can be assumed free of infection without individual testing.

Nucleic acid testing in minipools (of 6 to 24) is routinely used for screening for viruses (e.g., HBV, HIV) in donated blood after separating the whole blood to plasma or serum. However, plasma and serum cannot be used to detect pathogens of red blood cells. Additionally, whole blood cannot be pooled directly due to propensity for clotting when samples from different patients are mixed. Clots can also intefere with subsquent steps in the assay by altering the consistency of samples and clogging automated equipment.

### SUMMARY

Disclosed herein are methods for detecting a target in a sample aliquot comprising the steps of: providing a sample aliquot from each of a plurality of blood cell samples, thereby providing a plurality of sample aliquots; separately performing a lysing reaction on each of the sample aliquots, wherein the lysing step comprises contacting each of the sample aliquots with a lysis reagent comprising: a buffer, lithium lauryl sulfate (LLS), and at least one of a chloride containing salt and an anticoagulant selected from the group consisting of: EDTA, EDTA-Na₂, EGTA, and combinations thereof; wherein the reagent has a pH that is greater than 5.5, and whereby at least a portion of the blood cells in each of the sample aliquots lyse; combining the lysed sample aliquots into a single pooled sample to form a pooled lysate; separating a target from the pooled lysate; and performing a reaction to detect the separated target from the pooled lysate, wherein detection of the presence of the target in the pooled lysate indicates the presence of the target in at least one of the initially provided sample aliquots. In some embodiments, the methods further comprise, when the target is detected in the pooled lysate, individually testing the separate blood samples to identify which of the blood cell samples contain(s) the target. In some aspects, it is desired that the blood cell samples are free of the target, and as such samples determined to contain the target are discarded. In a non-limiting example of this aspect, a blood cell sample for use in blood banking is discarded if it is determined that a target is present in the sample. In some aspects, it is desired that the blood cell samples possess a target, and as such samples determined to contain the target are kept. In a non-limiting example of this aspect, a blood cell sample used for obtaining therapeutic targets is kept if it is determined that the target is present in the sample.

Disclosed herein are methods for separating a target from a plurality of sample aliquots, comprising the steps of: (a) providing a sample aliquot from each of a plurality of whole blood samples, thereby providing a plurality of sample aliquots; (b) separately performing a lysing reaction on each of the sample aliquots, wherein the lysing step comprises contacting each of the sample aliquots with a lysis reagent comprising (i) a buffer, (ii) lithium lauryl sulfate (LLS), and (iii) at least one of a chloride containing salt and an anticoagulant selected from the group consisting of: EDTA, EDTA-Na₂, EGTA, and combinations thereof; wherein the reagent has a pH that is greater than 5.5, and whereby at least a portion of the blood cells in each of the sample aliquots lyse; (c) combining the lysed sample aliquots into a single pooled sample to form a pooled lysate; and (d) separating a target from the pooled lysate. In some embodiments, the methods further comprise contacting the pooled lysate with a solid support configured to immobilize the target; and separating the immobilized target from the pooled lysate. In some embodiments, the target is a nucleic acid target and the solid support comprises an attached immobilized probe. In some embodiments wherein the target is a nucleic acid target, the methods further comprise contacting the pooled lysate with a capture probe comprising a first segment complementary to the nucleic acid target, and a second segment complementary to the immobilized probe. In some aspects, the methods further comprise providing hybridization conditions that favor formation of a hybridization complex between the first segment of the capture probe and the nucleic acid target. In some aspects, the methods further comprise providing hybridization conditions that favor formation of a hybridization complex between the second segment of the capture probe and the immobilized probe attached to the solid support. In some embodiments, the solid support is a magnetic bead solid support. In some embodiments, the solid support is a silica solid support. in some aspects, the silica solid support is glass wool. In some aspects, the silica solid support is a bead. In some embodiments, the solid support is contained within a column.

In one embodiment of the methods, the target is a nucleic acid target, and wherein the pooled lysate is tested for the presence or absence of the nucleic acid target using a nucleic acid amplification reaction and a detection reaction. In one aspect, the amplification reaction is an isothermal amplification reaction. In one aspect, the amplification reaction comprises performing a transcription mediated amplification reaction to generate an amplification product and wherein the detection reaction comprises detecting the amplification product with a detection probe. In one aspect, the amplification reaction and the detection reaction are performed simultaneously. In one embodiment, one or more steps of the methods are performed on an automated device. In one aspect, the amplification reaction and the detection reaction are performed on an automated device. In one aspect, the step of separating the target from the pooled lysate is performed on an automated device. In one aspect, the lysing reaction, the pooling reaction, the target separating reaction, and the nucleic acid analysis reaction are all performed on an automated device. In one aspect, the automated device is an integrated device that performes all of the automated steps. In one aspect, the automated device is a collection of spatially separated devices that each perform one or more of the automated steps.

In one embodiment of methods, the lysing reaction is performed on at least 4 sample aliquots. In one embodiment of methods, the lysing reaction is performed on at least 16 sample aliquots. In one embodiment of the methods, the lysing reaction is performed on at least 20 sample aliquots. In one embodiment of the methods, the lysing reaction is performed on up to 200 sample aliquots. In one embodiment of the methods, the lysing reaction is performed on from about 4 sample aliquots to about 200 aliquots.

In one embodiment of the methods, the buffer is sodium bicarbonate. In one aspect, the buffer is sodium bicarbonate and the reagent comprises a chloride containing salt that is ammonium chloride. In one aspect, the buffer is sodium bicarbonate, the reagent comprises a chloride containing salt that is ammonium chloride, and the pH of the reagent is from about 7.0 to about 8.0. In one aspect, the ammonium chloride is present in the reagent at a concentration from about 100 mM to about 500 mM, or at a concentration of about 250 mM. In one aspect, the sodium bicarbonate is present in the reagent at a concentration of about 5 mM to about 30 mM, or at a concentration of about 14 mM. In one aspect, the reagent comprises an anti-coagulant that is EDTA-Na₂ present in the reagent at a concentration of about 1 mM, EGTA present in the reagent at a concentration of about 1 mM, or is a combination of EDTA-Na₂ and EGTA, wherein the EDTA-NA₂ is present in the reagent at a concentration of about 1 mM and the EGTA is present in the reagent at a concentration of about 1 mM. In one aspect, the the lysis reagent comprises (i) about 14 mM sodium bicarbonate, (ii) about 5% (v/v) LLS, (iii) a chloride containing salt and an anticoagulant, wherein the chloride containing salt is ammonium chloride and the ammonium chloride is present in the reagent at a concentration of about 250 mM, and wherein the anti-coagulant is EDTA and the EDTA is present in the reagent at a concentration from about 0.1 mM to about 10 mM, and wherein the pH of the reagent is from about 7.2 to about 7.5. Ranges include all whole and partial numbers therein.

In one embodiment of the methods, the buffer is a TRIS buffer. In one embodiment, the reagent comprises a chloride salt that is magnesium chloride, and the magnesium chloride is present in the reagent at a concentration from about 20 mM to about 35 mM, or at a concentration of about 30 mM. In one aspect, the TRIS buffer is present in the reagent at a concentration from about 75 mM to about 150 mM, or at a concentration of about 100 mM. In one aspect, the reagent comprises a TRIS buffer and further comprises a chloride salt that is magnesium chloride, and the magnesium chloride is present in the reagent at a concentration from about 20 mM to about 35 mM, or at a concentration of about 30 mM. In one aspect, the lysis reagent comprises about 100 mM TRIS buffer and about 6% (v/v) LLS, and wherein the pH of the reagents is about 7.5. Ranges include all whole and partial numbers therein.

In one embodiment of the reagent, the LLS is present in the reagent at a concentration from about 4% (v/v) to about 15% (v/v), or at a concentration of about 10% (v/v), or at a concentration of about 8% (v/v), or at a concentration of about 6% (v/v), or at a concentration of about 5% (v/v). In one aspect, the buffer is sodium bicarbonate buffer. In one aspect, the buffer is TRIS buffer. Ranges include all whole or partial numbers therein.

In one embodiment, the reagent comprises a sodium phosphate buffer, about 10% (v/v) LLS, and an anti-coagulant, wherein the anti-coagulant is EDTA-Na₂ present in the reagent at a concentration of about 1 mM, EGTA present in the reagent at a concentration of about 1 mM, or is a combination of EDTA-Na₂ and EGTA, wherein the EDTA-NA₂ is present in the reagent at a concentration of about 1 mM and the EGTA is present in the reagent at a concentration of about 1 mM.

In one embodiment, the blood cell samples are whole blood samples. In one aspect, the blood cell samples are human whole blood samples. In one aspect, one or more of the blood cell samples are human whole blood samples and one or more of the blood cell samples are non-human whole blood samples. In one aspect, the blood cell samples contain red blood cells. In one aspect, the blood cell samples contain white blood cells.

In one embodiment, each sample aliquot is contacted with the lysis reagent at a volume ratio of sample aliquot to lysis reagent that is from about 1:2 to about 1:10 (v/v). In one aspect, the volume ratio of sample aliquot to lysis reagent is selected from the group consisting of: 1:2 (v/v), 1:3 (v/v), 1:4 (v/v), 1:5 (v/v), 1:6 (v/v), 1:7 (v/v), 1:8 (v/v), 1:9 (v/v), and 1:10 (v/v). In one aspect, the volume ratio of sample aliquot to lysis reagent is about 1:3 (v/v). In one aspect, the volume ratio of sample aliquot to lysis reagent is about 1:4 (v/v). Ranges include all whole and partial numbers therein.

In one embodiment, the target is a host-derived target. In one embodiment, the target is a pathogen-derived target. In one aspect, the target is released from a red blood cell. In one aspect, the target is a protein target. In one aspect, the target is a nucleic acid target. In one aspect, the target is an RNA target. In one aspect, the target is a ribosomal RNA target. In one aspect, the target is a pathogen derived target derived from a pathogen selected from the group consisting of: hepatitis viruses, human immunodeficiency viruses, dengue viruses, west nile viruses, flaviviruses, zika virus, and parasitic organisms. In one aspect, the target is a tpathogen-derived target derived from a parasitic organism selected from the group consisting of: parasites from the genus Babesia, parasites from the genus Plasmodium, parasites from the genus Trypanosoma, parasites from the genus Leishmania, parasites from the genus Anaplasma, parasites from the genus Toxoplasma, Babesia microti, Babesia divergens, Babesia duncani, Plasmodium falciparum, Plasmodium malariae, Plasmodium ovale, Plasmodium vivax, and Plasmodium knowlesi.

In one embodiment of the methods, wherein the target is a nucleic acid target, the separating step comprises contacting the pooled lysate with a capture probe and an immobilized probe, the capture probe having a first segment complementary to the nucleic acid target, and a second segment complementary to the immobilized probe, wherein the nucleic acid target binds to the capture probe, and wherein the bound capture probe binds to the immobilized probe. In one aspect, the immobilized probe is attached to a solid support. In one aspect, the pooled lysate is contacted with a solid support configured to immobilize the target; and separating the immobilized target from the pooled lysate. In one aspect, there are provided hybridization conditions that favor formation of a hybridization complex between the first segment of the capture probe and the nucleic acid target. In one aspect there are provided hybridization conditions that favor formation of a hybridization complex between the second segment of the capture probe and the immobilized probe attached to the solid support. In one aspect, the solid support is a magnetic bead solid support. In one aspect, the solid support is a silica solid support. In one aspect, the silica solid support is glass wool. In one aspect, the silica solid support is a bead. In one aspect, the solid support is contained within a column. In one aspect, the methods are performed without a centrifugation step.

In one embodiment, the entire volumes of the lysed sample aliquots are combined. In one aspect, 25% or less of volumes of the lysed sample aliquots are combined. In one aspect, at least 50% of the blood cells are lysed in five minutes or less after contacting the sample aliquot with the lysis reagent.

### DEFINITIONS

“Pathogens” include viruses, bacteria, protozoa, fungi, and other microorganisms responsible for disease in humans and other animals. Exemplary pathogens include, but are not limited to, hepatitis viruses, human immunodeficiency viruses, dengue viruses, west nile viruses, flaviviruses, zika virus, and parasitic organisms. Exemplary parasitic organisms include, but are not liminted to, parasites from the genus Babesia, parasites from the genus Plasmodium, parasites from the genus Trypanosoma, parasites from the genus Leishmania, parasites from the genus Anaplasma, parasites from the genus Toxoplasma, Babesia microti, Babesia divergens, Babesia duncani, Plasmodium falciparum, Plasmodium malariae, Plasmodium ovale, Plasmodium vivax, and Plasmodium knowlesi.

As used herein the term “target” can be a single type of molecule, such as a specific protein or nucleic acid from a pathogen or host cell. Alternatively, a target can be a class of molecules such as any protein target or nucleic acid target from a pathogen or human cell. For example, a target that is a single type of nucleic acid target from a pathogen can be a ribosomal RNA target from a parasite, such as 18S rRNA target from a Babesia organism, or a target that is a class of nucleic acid from a pathogen can be all RNA from a whole blood sample suspected of being infected by a pathogen. Targets from host cells can be therapeutic targets, such as, for example, an antibody or a protein (such as a clotting factor). Multiple distinct targets can also be analyzed, such as an RNA target and a protein target, or two distinct RNA targets, such as two different mRNA targets, or an mRNA target and an rRNA target. Targets include endogenous components of the host's blood cells (host-derived targets) and components arising as a result of pathogenic infection of infected red blood cells and are typically encoded by the infecting pathogen (i.e., “pathogenic” or “pathogen-derived” targets).

A “lysis reagent” as referred to herein, is a reagent, often provided in the form of a solution, effective for inducing lysis of blood cells in a sample, including whole blood samples, samples containing red blood cells, samples containing white blood cells and sample containing red blood cell products such as pelleted red blood cells. In some instances, the lysis reagent preferentially lyses red blood cells. Preferential lysis of red blood cells over other cellular components of blood means that the percentage of red blood cells lysed is higher than that of other cellular components present in the sample being analyzed, the other cell types being assessed in the aggregate. A number of lysis reagents are useful for the lysate pooling methods described herein. Preferred lysis reagents are comprised of: a buffer, lithium lauryl sulfate (LLS), and at least one of a chloride containing salt and an anticoagulant selected from the group consisting of: EDTA, EDTA-Na₂, EGTA, and combinations thereof; wherein the reagent has a pH that is greater than 5.5.

As used herein, a “sample aliquot” refers to a smaller volume of a sample. Typically, the sample aliquot is removed from the larger sample volume for testing. Each of the sample aliquots are preferably separately lysed using a lysis reagent as described herein. Lysed sample aliquots are then combined to form a pooled lysate. The pooled lysate contains any target present in one or more of the now lysed sample aliquots, along with other components such as non-target material released from within the blood cells, and unlysed blood cells. As used herein, a plurality of sample aliquots refers to two or more sample aliquots, each of the sample aliquots being pulled from two or more samples. The sample aliquots are preferably not combined until after the sample aliquots are mixed with the lysis reagent under conditions to promote lysis of at least a portion of the blood cells in the sample aliquot.

Anionic detergents are compounds with a negatively charged, anionic head group and a long hydrocarbon tail, often provided as a salt with an alkali metal or ammonium ion.

Anti-coagulants inhibit clotting of whole blood. Anti-coagulants include heparins and calcium chelating agents. Heparins activate antithrombin III, which inhibits the activity of thrombin and other proteases involved in blood clotting. Calcium chelating agents, such as EDTA (Ethylenedinitrilo)tetraacetic acid), EDTA-Na2 (Disodium ethylenediaminetetraacetate dihydrate), EGTA (Ethylene-bis(oxyethylenenitrilo)tetraacetic acid), and citrate, bind calcium ions required for blood clotting.

A buffer refers to a weak acid or weak base used to maintain the pH of a solution. Preferred buffers for use herein include, but are not limited to sodium bicarbonate, TRIS (2-Amino-2-(hydroxymethyl)-1,3-propanediol), sodium phosphate dibasic, and sodium phosphate monobasic.

A nucleic acid refers to a multimeric compound comprising nucleotides or analogs that have nitrogenous heterocyclic bases or base analogs linked together to form a polymer, including conventional RNA, DNA, mixed RNA-DNA, and analogs thereof.

The nitrogenous heterocyclic bases can be referred to as nucleobases. Nucleobases can be conventional DNA or RNA bases (A, G, C, T, U), base analogs, and others (See e.g., The Biochemistry of the Nucleic Acids 5-36; Adams et al., ed., 11.sup.th ed., 1992; van Aerschott et al., 1995, Nucl. Acids Res. 23(21): 4363-70; Nair et al., 2001, Nucleosides Nucleotides Nucl. Acids, 20(4-7):735-8; Hill et al., 1998, Proc. Natl. Acad. Sci. USA 95(8):4258-63; Lin and Brown, 1992, Nucl. Acids Res. 20(19):5149-52; Okamoto et al., 2002, Bioorg. Med. Chem. Lett. 12(1):97-9; Nguyen et al., 1998, Nucl. Acids Res. 26(18):4249-58; Kiopffer & Engels, 2005, Nucleosides Nucleotides Nucl. Acids, 24(5-7) 651-4; Babu & Wengel, 2001, Chem. Commun. (Camb.) 20: 2114-5; Hrdlicka et al., 2005, J. Am. Chem. Soc. 127(38): 13293-9; U.S. Pat. No. 5,378,825; WO 93/13121; Gamper et al., 2004, Biochem. 43(31): 10224-36; and Berger et al., 2000, Nucl. Acids Res. 28(15): 2911-4). Many derivatized and modified nucleobases or analogues are commercially available (e.g., Glen Research, Sterling, Va.).

A nucleobase unit attached to a sugar, can be referred to as a nucleobase unit, or monomer. Sugar moieties of a nucleic acid can be ribose, deoxyribose, or similar compounds, e.g., with 2′ methoxy or 2′ halide substitutions. Nucleotides and nucleosides are examples of nucleobase units. The nucleobase units can be joined by a variety of linkages or conformations, including phosphodiester, phosphorothioate or methylphosphonate linkages, peptide-nucleic acid linkages (PNA; Nielsen et al., 1994, Bioconj. Chem. 5(1): 3-7; PCT No. WO 95/32305), and a locked nucleic acid (LNA) conformation in which nucleotide monomers with a bicyclic furanose unit are locked in an RNA mimicking sugar conformation (Vester et al., 2004, Biochemistry 43(42):13233-41; Hakansson & Wengel, 2001, Bioorg. Med. Chem. Lett. 11 (7):935-8), or combinations of such linkages in a nucleic acid strand. Nucleic acids may include one or more “abasic” residues, i.e., the backbone includes no nitrogenous base for one or more positions (U.S. Pat. No. 5,585,481).

A nucleic acid may include only conventional RNA or DNA sugars, bases and linkages, or may include both conventional components and substitutions (e.g., conventional RNA bases with 2′-O-methyl linkages, or a mixture of conventional bases and analogs). Inclusion of PNA, 2′-methoxy or 2′-fluoro substituted RNA, or structures that affect the overall charge, charge density, or steric associations of a hybridization complex, including oligomers that contain charged linkages (e.g., phosphorothioates) or neutral groups (e.g., methylphosphonates) may affect the stability of duplexes formed by nucleic acids.

An oligomer may contain a “random polymer” sequence that refers to a population of oligomers that are substantially the same in overall length and other characteristics, but in which at least a portion of the oligomer is synthesized by random incorporation of different bases for a specified length, e.g., a random assortment of all four standard bases (A, T, G, and C) in a DNA oligomer, or a random assortment of a few bases (U or T and G) in a defined portion of a larger oligomer. The resulting oligomer is actually a population of oligomers whose finite number of members is determined by the length and number of bases making up the random portion (e.g., 2⁶ oligomers in a population of oligomers that contains a 6-nt random sequence synthesized by using 2 different bases).

Complementarity of nucleic acids means that a nucleotide sequence in one strand of nucleic acid, due to orientation of its nucleobase groups, hydrogen bonds to another sequence on an opposing nucleic acid strand. The complementary bases typically are, in DNA, A with T and C with G, and, in RNA, C with G, and U with A. Complementarity can be perfect (i.e., exact) or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm of hybridized strands, or by empirical determination of Tm by using routine methods. Tm refers to the temperature at which a population of hybridization complexes formed between two nucleic acid strands are 50% denatured. At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

“Separating” or “isolating” or “purifying” refers to removing one or more components from a complex mixture, such as a sample. Preferably, a separating, isolating or purifying step removes at least 70%, preferably at least 90%, and more preferably at least 95% w/w of the target nucleic acids from other sample components. A separating, isolating or purifying step may optionally include additional washing steps to remove non-target sample components.

“Release” of a capture hybrid refers to separating one or more components of a capture hybrid from each other, such as separating a target nucleic acid from a capture probe, and/or a capture probe from an immobilized probe. Release of the target nucleic acid strand separates the target from other components of a capture hybrid and makes the target available for binding to a detection probe. Other components of the capture hybrid may remain bound, e.g., the capture probe strand to the immobilized probe on a capture support, without affecting target detection.

A “label” refers to a molecular moiety that is detectable or produces a detectable response or signal directly or indirectly, e.g., by catalyzing a reaction that produces a detectable signal. Labels include luminescent moieties (such as fluorescent, bioluminescent, or chemiluminescent compounds), radioisotopes, members of specific binding pairs (e.g., biotin and avidin), enzyme or enzyme substrate, reactive groups, or chromophores, such as a dye or particle that results in detectable color.

A “capture probe” includes a first segment including a target-complementary region of sequence and a second segment for attaching the capture probe, or a hybridization complex that includes the capture probe, to an immobilized probe. The first segment can be configured to substantially complementary to a specific target nucleic acid sequence so that a first segment and a target nucleic acid can hybridize to form a stable duplex (i.e., having a detectable melting point) under hybridizing conditions, such as described in the Examples. Alternatively, the first segment can be configured to nonspecifically bind to nucleic acid sequences in a sample under hybridizing conditions (see WO 2008/016988). The second segment includes a region of sequence that is complementary to a sequence of an immobilized probe. Preferably, a chimeric capture probe includes a nucleic acid homopolymer (e.g., poly-A or poly-T) that is covalently attached to the target-complementary region of the capture probe and that hybridizes under appropriate conditions to a complementary homopolymer of the immobilized probe (e.g., poly-T or poly-A, respectively) as previously described (U.S. Pat. No. 6,110,678 to Weisburg et al.). Capture probes may further comprise a third segment that acts as a closing sequence to inactivate unbound target capture probes in a capture reaction. This third segment can flank the first segment opposite the second segment (e.g., capture sequence:target hybridizing sequence:closing sequence) or it can flank the second segment opposite the first segment (e.g., closing sequence:capture sequence:target hybridizing sequence). See WO 2006/007567 and US 2009-0286249.

An “immobilized probe” includes a nucleic acid joined directly or indirectly to a support. The nucleic acid is complementary to a nucleic acid in the capture probe, although may or may not be the same length (number of nucleobase units) as the in the capture probe. The nucleic acid in the immobilized probe preferably contains at least six contiguous nucleobase units and can contain for example 10-45 or 10-40 or 10-30 or 10-25 or 15-25, inclusively, L-nucleobase units. The nucleic acid is preferably a homopolymer, and more preferably a homopolymer of adenine or thymine. A preferred form of immobilized probe is or includes a homopolymer of 14 thymine residues for use in combination with a capture probe including a second segment with a homopolymer of adenine residues. The nucleic acid moiety of an immobilized probe is typically provided in single-stranded form, or if not, is denatured to single-stranded form before or during use.

As used herein, a “solid support” is any of a variety of materials that are useful as a support for the immobilized probes, e.g., matrices or particles made of nitrocellulose, nylon, silica, polyacrylate, mixed polymers, polystyrene, silane polypropylene, and magnetically attractable materials. Solid supports in the shape of a sheet, a bead/sphere, awool/fiber and other common shapes are useful with the methods. Monodisperse magnetic beads are a preferred support because they are relatively uniform in size and readily retrieved from solution by applying a magnetic force to the reaction container, preferably in an automated system. The solid supports may be broadly dispursed in a slurry or they may be packed into a column. An immobilized probe may be linked directly to the capture support, e.g., by using any of a variety of covalent linkages, chelation, or ionic interaction, or may be linked indirectly via one or more linkers joined to the support. The linker can include one or more nucleotides not intended to hybridize to the capture probe but to act as a spacer between the nucleic acid of the immobilized probe and its support.

A “detection probe” is a nucleic acid or other molecule that binds specifically to a target sequence and which binding results, directly or indirectly, in a detectable signal to indicate the presence of the target sequence. A detection probe need not be labeled to produce a detectable signal, e.g., an electrical impulse resulting from binding the probe to its target sequence may be the detectable signal. A “labeled probe” is a probe that contains or is linked, directly or indirectly, to a label (e.g., Sambrook et al., Molecular Cloning, A Laboratory Manual, 2nd ed., Chapt. 10; U.S. Pat. No. 6,361,945, Becker et al.; U.S. Pat. No. 5,658,737, Nelson et al.; U.S. Pat. No. 5,656,207, Woodhead et al.; U.S. Pat. No. 5,547,842, Hogan et al.; U.S. Pat. No. 5,283,174, Arnold et al.; U.S. Pat. No. 4,581,333, Kourilsky et al.; U.S. Pat. No. 5,731,148, Becker et al.). For example, detection probes may include a non-nucleotide linker and a chemiluminescent label attached to the linker (U.S. Pat. Nos. 5,185,439, 5,585,481 and 5,639,604, Arnold et al.). Examples of detection probes include oligonucleotides of about 5 to 50 nucleotides in length having an attached label that is detected in a homogeneous reaction, e.g., one that uses differential hydrolysis of a label on a bound or unbound probe.

Detection probes can have a nucleotide sequence that is of the same or opposite sense as a target sequence depending on the format of the assay. Detection probes can hybridize to the same or different segment of a target sequence as a capture probe. Some detection probes have an attached chemiluminescent marker, e.g., an acridinium ester (AE) compound (U.S. Pat. Nos. 5,185,439, 5,639,604, 5,585,481, and 5,656,744). In some detection probes, an acridinium ester label is attached to a central region of the probe near a region of A and T/U base pairs by using a non-nucleotide linker (U.S. Pat. Nos. 5,585,481 and 5,656,744, Arnold, et al.) which restricts the amines of the nucleotide bases on both sides of the AE and provides a site for intercalation. Alternatively, an AE label may be attached to the 3′ or 5′ terminus of the detection probe which is used in conjunction with a second oligomer that hybridizes adjacent to the detection probe on the target nucleic acid to restrict the effects of nearby amine contributed by the target nucleic acid. In some detection probes, an AE label at or near the site of a mismatch with a related non-target polynucleotide sequence, to permit discrimination between the related sequence and the target sequence that may differ by only one nucleotide because the area of the duplex around the mismatch site is sufficiently destabilized to render the AE on the probe hybridized to the related non-target sequence susceptible to hydrolysis degradation. HIV-1 and HCV may be detected using a modified form of the commercial PROCLEIX® ULTRIO assay from Grifols International, S.A. (Spain). The modification involves replacing the D-polyA and D-polyT sequences in capture and immobilized probes with L-poly A and L-poly-T, respectively.

“Hybridization condition” refers to the cumulative environment in which one nucleic acid strand bonds to a second nucleic acid strand by complementary strand interactions and hydrogen bonding to produce a hybridization complex. Such conditions include the chemical components and their concentrations (e.g., salts, chelating agents, formamide) of an aqueous or organic solution containing the nucleic acids, and the temperature of the mixture. Other factors, such as the length of incubation time or reaction chamber dimensions may contribute to the environment (e.g., Sambrook et al., Molecular Cloning, A Laboratory Manual, 2.sup.nd ed., pp. 1.90-1.91, 9.47-9.51, 11.47-11.57 (Cold Spring Harbor Laboratory Press, Cold Spring Harbor, N.Y., 1989)).

Specific binding of a target capture oligomer to a target nucleic or target nucleic acids means binding between a single defined sequence in the first segment of a target capture oligomer and an exactly or substantially complementary segment on target nucleic acid(s) to form a stable duplex. Such binding is detectably stronger (higher signal or melting temperature) than binding to other nucleic acids in the sample lacking a segment exactly or substantially complementary to the single defined target capture oligomer sequence. Non-specific binding of a target capture oligomer to target nucleic acids means that the target capture oligomer can bind to a population of target sequences that do not share a segment having exact or substantial complementarity to a single defined target capture oligomer sequence. Such can be achieved by for example using a randomized sequence in the first segment of the capture probe.

“Release” of a capture hybrid refers to separating one or more components of a capture hybrid from each other, such as separating a target nucleic acid from a capture probe, and/or a target capture oligomer from an immobilized probe. Release of the target nucleic acid strand separates the target from other components of a capture hybrid and makes the target available for binding to a detection probe. Other components of the capture hybrid may remain bound, e.g., the target capture oligomer strand to the immobilized probe on a capture support, without affecting target detection.

“Sensitivity” is the proportion of true positives correctly identified as such (e.g. the percentage of infected blood samples as having the infection). Sensitivity can also be characterized by a lower limit of detection. For example, at pathogen concentration of 1 particle per mL blood before pooling, an assay detects at least 95% of infected samples. The lower the limit of detection, the higher the sensitivity of the assay. Specificity measures the proportion of true negatives which are correctly identified (e.g. the percentage of uninfected blood samples correctly identified as not having the infection.)

Reference to a range of values also includes integers within the range and sub-ranges defined by integers in the range. Reference to any numerical value or range of numerical values should be understood as encompassing any such variation as is inherent in measuring that value other typical conditions of use.

### DETAILED DESCRIPTION

### I. General

The disclosure provides methods of pooled analysis of samples containing blood cells, such as whole blood cell samples. The methods are particularly useful for screening whole blood samples collected from many individuals to be used for transfusions and the like. Before pooling, aliquots of such samples are individually contacted with a lysis reagent, further described below, to lyse red blood cells in the samples. After pooling, the combined lysate or a specimen thereof is tested for presence of target(s) characteristic of pathogen(s). By lysing prior to pooling, multiple target molecules (e.g., RNA) can be released prior to pooling, increasing the probability that an infected sample will be detected against a background of uninfected samples with which it is pooled. The method are particularly useful for identifying pathogens of red blood cells, but can also be used to run a complete panel of testing including at least for all of the typical targets described in the Background.

### II. Lysis Reagents

The lysis reagent, which was first described in copending application WO 2016/064887 , filed Oct. 20, 2015, incorporated by reference, includes at least a buffer, lithium lauryl sulfate (LLS), and at least one of a chloride containing salt and an anticoagulant selected from the group consisting of: EDTA, EDTA-Na2, EGTA, and combinations thereof.

In some embodiments, the buffer is a sodium bicarbonate buffer. In some embodiments, the buffer is a TRIS (2-Amino-2-(hydroxymethyl)-1,3-propanediol) buffer. In some embodiments, the buffer is a sodium bicarbonate buffer. In some embodiments, the buffer is a sodium phosphate buffer. In some embodiments, the buffer is a sodium bicarbonate buffer in the reagent in a concentration from about 5 mM to about 30 mM, from about 10 mM to about 20 mM, about 10 mM to about 15 mM, or from about 15 mM to about 20 mM. In some embodiments, the buffer is a TRIS buffer in the reagent at a concentration from about 75 mM to about 150 mM, from about 75 mM to about 125 mM, from about 100 mM to about 125 mM, or from about 90 mM to about 110 mM. In some embodiments, the buffer is a sodium phosphate (Na₃PO₄) buffer in the reagent at a concentration from about 5 mM to about 30 mM, from about 10 mM to about 20 mM, about 10 mM to about 15 mM, or from about 15 mM to about 20 mM. In some embodiments, the concentration of sodium phosphate in the reagent is from about 8 mM to about 40 mM, from about 10 mM to about 33 mM, from about 15 mM to about 30 mM, about 30 mM, or about 15 mM. In some embodiments, the concentration of sodium phosphate monobasic in the reagent is from about 8 mM to about 40 mM, from about 10 mM to about 33 mM, from about 15 mM to about 30 mM, about 30 mM, or about 15 mM. In some embodiments, the concentration of sodium phosphate dibasic in the reagent is from about 8 mM to about 40 mM, from about 10 mM to about 33 mM, from about 15 mM to about 30 mM, about 30 mM, or about 15 mM. Ranges include all whole and partial numbers therein.

In some embodiments, the anti-coagulant is one or more of EDTA ((Ethylenedinitrilo)tetraacetic acid), EDTA-Na₂ (Disodium ethylenediaminetetraacetate dihydrate), EGTA (Ethylene-bis(oxyethylenenitrilo)tetraacetic acid), heparin, or citrate. In some embodiments, the anti-coagulant comprises an EDTA in the reagent at a concentration from about 0.05 mM to about 15 mM, from about 0.1 mM to about 10 mM, or from about 0.5 mM to about 5 mM. In some embodiments, the anti-coagulant is EDTA in the reagent at a concentration from about 0.05 mM to about 15 mM, from about 0.1 mM to about 10 mM, or from about 0.5 mM to about 5 mM. In some embodiments, the anti-coagulant is EDTA-Na₂ in the reagent at a concentration from about 0.05 mM to about 15 mM, from about 0.1 mM to about 10 mM, or from about 0.5 mM to about 5 mM. In some embodiments, the anti-coagulant is EGTA in the reagent at a concentration from about 0.05 mM to about 15 mM, from about 0.1 mM to about 10 mM, or from about 0.5 mM to about 5 mM. Ranges include all whole and partial numbers therein.

In some embodiments, the salt comprises one or more of the following ions: a sodium ion, a potassium ion, an ammonium ion, a magnesium ion, a lithium ion, and a chloride ion. In some embodiments, the salt is magnesium chloride, ammonium chloride, potassium chloride, or sodium chloride. In some embodiments, the salt comprises a chloride ion and one of a magnesium ion, sodium ion or potassium ion, and the concentration of the salt in the reagent is from about 10 mM to about 50 mM, from about 15 mM to about 40 mM, or from about 20 mM to about 35 mM. In some embodiments, the salt is ammonium chloride in the reagent at a concentration from about 100 mM to about 500 mM, from about 200 mM to about 350 mM, or from about 250 mM to about 300 mM. Ranges include all whole and partial numbers therein.

In some embodiments, the detergent is one of lithium lauryl sulfate (LLS), nonyl phenoxypolyethoxylethanol (NP 40), sodium dodecyl sulfate (SDS), and Triton-X 100. In some embodiments, the detergent is an anionic detergent. In some embodiments, the detergent is LLS or SDS. In some embodiments, the detergent is present in the reagent at a concentration that is greater than about 1.5% (v/v). In some embodiments, the detergent is present in the reagent at a concentration that is less than about 15.5% (v/v). In some embodiments, the detergent is present in the reagent at a concentration of from about 2% to about 15% (v/v). In some embodiments, the detergent is present in the reagent at a concentration from about 2% to about 15% (v/v). In some embodiments, the detergent is LLS and the concentration of LLS in the reagent is from about 2% to about 15% (v/v), from about 4% to about 10% (v/v), or from about 5% to about 8% (v/v). In some embodiments, the detergent is LLS and is present in the reagent at about 14 mM to about 50 mM. Ranges include all whole and partial numbers therein.

In some embodiments, the pH of the reagent is greater than a pH of 5.5. In some embodiments, the pH of the reagent is less than a pH of 10.5. In some embodiments, the pH of the reagent is from about 6.0 to about 10.0. In some embodiments, the pH of the reagent is from about 6.5 to about 8.0, or from about 7.0 to about 8.0, or from about 7.2 to about 7.6, or from about 6.7 to about 7.5, or about 6.7, or about 7.3 or about 7.5. Ranges include all whole and partial numbers therein.

In some embodiments of the reagent, the concentration of sodium bicarbonate is 14 mM, the concentration of ammonium chloride is 250 mM, the concentration of LLS is 8% (v/v), the concentration of EDTA is from about 0.1 mM to about 10 mM, and the pH is 7.2-7.6. In some embodiments of the reagent, the buffer is selected from the group consisting of sodium bicarbonate, sodium phosphate and TRIS, the detergent is from about 5% to about 10% (v/v), the pH is from about 6.5 to about 8.0, and the salt is selected from the group consisting of magnesium chloride, ammonium chloride, and potassium chloride. In some embodiments of the reagent, the buffer is selected from the group consisting of sodium phosphate and TRIS and the salt is selected from the group consisting of magnesium chloride and ammonium chloride. In some aspects of this embodiment, the concentration of anti-coagulant is about 0 mM to about 1 mM. In some further aspects of this embodiment, the detergent is LLS at a concentration from about 6% to about 10% (v/v). In some further aspects of this embodiment, the buffer is TRIS at a concentration from about 90 mM to about 110 mM and the pH of the reagent is from about 7.2 to about 7.5. In some further aspects of this embodiment, the anti-coagulant is at a concentration of about 0.1 mM to about 5 mM and is EGTA, EDTA, EDTA-Na₂ or a combination thereof.

The lysis reagent serves to lyse blood cells, protect a released target from degradation in the lysate by nucleases or proteases, and is compatible with subsequent steps for use of the target, such as target capture, amplification, detection, and/or sequencing. The lysis reagent is particularly amenable for analysis of RNA from pathogens infecting red blood cells, including parasitic organisms such as Babesia and Plasmodium species. The lysis reagent is compatible with methods of nucleic acid detection without removal of the lysis reagent from samples

### III. Use of Lysis Reagents

Blood cells can be obtained from any available source, such as whole blood or any fraction thereof including those containing red blood cells, such as pelleted red blood cells. Whole blood can be human whole blood, or non-human whole blood, or a combination thereof.

The lysis reagent can be admixed with blood cells for a time sufficient to induce cell lysis and cause release of the desired target(s) from cells. Exemplary times for maintaining blood cells admixed with lysis reagent include 1-30 minutes, 2-15 minutes, 3-10 minutes, 4-6 minutes, or 5 minutes. Preferably, the time is at least 5 minutes. Preferably the mixture lacks visible particles after lysis.

The temperature of incubation of the lysis reagent with blood cells can vary. The temperature is preferably chosen to maximize extent and rate of lysis to minimize degradation of target(s) or prevent inhibition of subsequent processing. Exemplary temperature ranges include 0-50° C., 5-45° C., 10-40° C., 15-37° C., 20-30° C., 22-27° C., or 25° C. Ambient temperature is suitable. Lysis of blood cells should release a sufficient amount of target molecules to be detectable by the methods described herein. Preferably lysis results in at least 50%, 60%, 70%, 80%, 90%, or 100% lysis of blood cells in a sample being lysed. Ranges include all whole and partial numbers therein.

The ratio at which a blood cell sample is combined with lysis reagent can affect the extent and rate of cell lysis and protection of target molecules from degradation after release from lysed cells. Exemplary ratios in which a blood cell sample is admixed with the lysis reagent include ratios of about 1:1, 1:2, 1:3, 1:4, 1:5, 1:6, 1:7, 1:8, 1:9, 1:10, or in a range of ratios between 1:1 and 1:5 or 1:10 (v/v; sample:reagent). A preferred ratio is a sample aliquot admixed with the lysis reagent at a ratio of about 1:3 (v/v; sample:reagent). Another preferred ratio is a sample aliquot admixed with the lysis reagent at a ratio of about 1:4 (v/v; sample:reagent). When the sample comprises red blood cells isolated from whole blood, such as pelleted red blood cells, the red blood cells can be admixed with the lysis reagent at exemplary ratios of 1:1, 1:2, 1:3, 1:4, 1:5, 1:10, or in a range of ratios between 1:1 and 1:10 (v/v; red blood cells:reagent). Ranges include all whole and partial numbers therein.

### IV. Targets

Targets released from blood cells by the present lysis reagent can include host-derived targets and pathogen derived targets. Targets include protein targets (e.g., peptide targets and antibody targets), nucleic acid targets (e.g., DNA target or RNA target), whole particles, lipids and carbohydrates. The RNA targets can be ribosomal RNA (rRNA) targets, messenger RNA (mRNA) targets, or heterogeneous nuclear RNA (hnRNA) targets. A preferred target for pathogen-derived targets is a ribosomal RNA target, particularly 18S rRNA targets, 5S rRNA targets, 5.8S rRNA targets, or 28S rRNA targets. Targets can be released from white blood cells, red blood cells or be present in plasma. Targets include therapeutic targets. Therapeutic targets are preferably host derived targets (such as peptide targets (e.g., thrombin) and antibody targets).

Exemplary pathogenic organisms include hepatitis viruses, human immunodeficiency viruses, dengue viruses, west nile viruses, flaviviruses, zika virus, and parasitic organisms. Exemplary parasitic organisms include parasites from the genus Babesia, parasites from the genus Plasmodium, parasites from the genus Trypanosoma, parasites from the genus Leishmania, parasites from the genus Anaplasma, parasites from the genus Toxoplasma, Babesia microti, Babesia divergens, Babesia duncani, Plasmodium falciparum, Plasmodium malariae, Plasmodium ovale, Plasmodium vivax, and Plasmodium knowlesi.

### V. Assays

Target molecules released from lysis of a sample aliquot containing blood cells are subject to analysis. Target molecules may or may not be separated from the lysis reagent (by centrifugation or otherwise) before analysis. Omission of a separation step can facilitate efficient work flow in performing the assay. The type of assay depends on the targets. The assay can be a real-time or end-point assay.

A. Nucleic Acids

Analysis of nucleic acid targets often involves steps of target capture, amplification and detection. Alternatively, amplification and detection methods can be performed without prior target capture. Preferably amplification, and detection and target capture (if performed) occur without separation of target molecules from the lysis reagent. Thus, the entire process can be performed in a single vessel.

1. Target Capture Assay

An exemplary target capture assay can be performed as follows using one or more capture probes, an immobilized probe, a sample, and a suitable medium to permit hybridization of the target capture oligomer to the target nucleic acid and of target capture oligomer to the immobilized probe. The target sample (preferably herein the target sample is the pooled lysate) can be heated (e.g., from 65° C. to 95° C.) before performing the assay to denature any nucleic acids in double-stranded form. The components can be mixed in any order. For example the target capture oligomer can be added to the target sample and hybridized with the nucleic acid target in the target sample before adding the immobilized probe. However, for an automated assay, it is preferable to minimize the number of adding steps by supplying the target capture oligomer and immobilized probe at the same or substantially the same time. In this case, the order of hybridization can be controlled by performing a first hybridization under conditions in which a duplex can form between the first segment of the target capture oligomer and the nucleic acid target but which exceeds the melting temperature of the duplex that would form between first and second segments of the capture probe and between the target capture oligomer and immobilized probe. Next is performed a second hybridization under conditions of reduced stringency, preferably below the melting temperature of the duplexes formed between the first segment of the target capture oligomer the nucleic acid target. Under the second hybridization conditions a duplex can form between the second segment of the capture probe and the immobilized probe. Stringency can be reduced by lowering the temperature of the assay mix. For example, the higher stringency hybridization can be performed at or around 60° C. and the lower stringency hybridization by allowing cooling to room temperature or 25° C. Stringency can also be reduced by reducing salt concentration or adding or increasing concentration of a chaotropic solvent. In some methods, all steps (with the possible exception of an initial denaturation step at higher temperature to denature double stranded target) can be performed isothermally.

Following formation of the nucleic acid target:capture probe:immobilized probe hybrid (the capture complex), the capture complex is separated away from other sample components. One method for separating the capture complex from other sample components is by physically separating the solid support comprising the capture complex from other sample components by using any of a variety of known methods, e.g., centrifugation, filtration, or magnetic attraction of a magnetic capture support. The separation is preferably performed at a temperature below the melting temperature of the duplexes formed between the nucleic acid target, the target capture oligomer, and the immobilized probe. In some methods, the separation is performed at a temperature less than but within 10° C. of the melting temperature of the stem-loop structure (e.g., at 60° C.) to maintain stringency of hybridization conditions and consequent ability to distinguished matched and unmatched target nucleic acids. Another method includes allowing the capture complex to remain attached to a solid support placed in a column while the sample components elute out of the column and into one or more containers. The column comprising the capture complex is then subjected to eluting conditions to allow the captures nucleic acid target to elute into a separate container. ((See e.g., U.S. Pat. No. 6,110,678)

To further facilitate isolation of the target nucleic acid from other sample components that adhere non-specifically to any portion of the capture hybrid, the capture hybrid may be washed one or more times to dilute and remove other sample components. Washing may be accomplished by dissociating the capture hybrid into its individual components in an appropriate aqueous solution (e.g., a solution containing Tris and EDTA. See e.g., U.S. Pat. No. 6,110,678) and appropriate conditions (e.g., temperature above the Tₘ of the components) and then readjusting the conditions to permit reformation of the capture hybrid. However, for ease of handling and minimization of steps, washing preferably rinses the intact capture hybrid attached to the capture support in a solution by using conditions that maintain the capture hybrid. Preferably, capture of the target nucleic acid with washing, if performed, isolates at least 70%, preferably at least 90%, and more preferably about 95% of the target nucleic acids away from other sample components. Isolated nucleic acids can be used for a number of downstream processes, such as nucleic acid amplification.

A target capture assay may also be performed as part of a real-time, biphasic, target capture and amplification method. In such a method, 500 μL of sample and 400 μL of target capture reagent (TCR) are added to reaction tubes. The TCR contains magnetic particles, components to lyse organisms present in the sample, capture oligos, a T7 initiation promoter, and an internal calibrator. Fluid in the reaction tubes is mixed for a specific time and speed to ensure the mixture is homogeneous. Reaction tubes are then transferred to a transition incubator at 43.7° C. to preheat the fluid in the reaction tubes. Reaction tubes are then transferred to an anneal incubator set at 64° C. During incubation at 64° C., any organisms present in the sample that were not previously disrupted by the lysis reagent are disrupted, causing release of the target. Reaction tubes are then moved to a transition incubator to start a cool down process, and are further cooled in a chiller ramp (17° C. to 19° C.), leading to binding of the T7 initiation promoter and capture of both the target and the internal calibrator to the magnetic particles via the capture oligos. The reaction tubes are moved to a magnetic parking station where they are subjected to magnets which pull the magnetic particles to the sides of the tubes prior to entering a wash station. In the wash station, potential interfering substances are removed from the reaction by washing the magnetic particles.

2. Amplification

A nucleic acid analyte can be amplified using methods such as isothermal amplification reactions (e.g., transcription mediated amplification (TMA), nucleic acid sequence based amplification (NASBA), loop mediated isothermal amplification, polymerase spiral reaction (PSR) (Liu, W. et al. Polymerase Spiral Reaction (PSR): A novel isothermal nucleic acid amplification method. Sci. Rep. 5, 12723; (2015)), ligase chain reaction, and other isothermal amplification methods), or temperature cycling amplification reactions (e.g., polymerase chain reaction (PCR), quantitative PCR (qPCT), real time PCR (rt-PCT), or other temperature cycling amplification methods), or other amplification methods. Detection of the amplified RNA analyte products can be performed during amplification (real-time) or following amplification (end-point).

### i. Transcription Mediated Amplification

TMA has been previously described (e.g., U.S. Pat. Nos. 5,399,491, 5,554,516, 5,824,518 and 7,833,716; and also e.g., F. Gonzales and S. McDonough. Applications of Transcription-Mediated Amplification to Quantification of Gene Sequences. Gene Amplification. 1998 Ed. Francois Ferre, Birkhauser, Boston. PP. 189-204). In TMA, a target nucleic acid that contains the sequence to be amplified is provided as single stranded nucleic acid (e.g., ssRNA or ssDNA). Any conventional method of converting a double stranded nucleic acid (e.g., dsDNA) to a single-stranded nucleic acid may be used. A promoter primer binds specifically to the target nucleic acid at its target sequence and a reverse transcriptase (RT) extends the 3′ end of the promoter primer using the target strand as a template to create a cDNA copy, resulting in a RNA:cDNA duplex. RNase activity (e.g., RNase H of RT enzyme) digests the RNA of the RNA:cDNA duplex and a second primer binds specifically to its target sequence in the cDNA, downstream from the promoter-primer end. Then RT synthesizes a new DNA strand by extending the 3′ end of the second primer using the cDNA as a template to create a dsDNA that contains a functional promoter sequence. RNA polymerase specific for the functional promoter initiates transcription to produce about 100 to 1000 RNA transcripts (amplified copies or amplicons) complementary to the initial target strand. The second primer binds specifically to its target sequence in each amplicon and RT creates a cDNA from the amplicon RNA template to produce a RNA:cDNA duplex. RNase digests the amplicon RNA from the RNA:cDNA duplex and the target-specific sequence of the promoter primer binds to its complementary sequence in the newly synthesized DNA and RT extends the 3′ end of the promoter primer as well as the 3′ end of the cDNA to create a dsDNA that contains a functional promoter to which the RNA polymerase binds and transcribes additional amplicons that are complementary to the target strand. Autocatalytic cycles that use these steps repeatedly during the reaction produce about a billion-fold amplification of the initial target sequence. Optionally, amplicons may be detected during amplification (real-time detection) or at an end point of the reaction (end-point detection) by using a probe that binds specifically to a sequence contained in the amplicons. Detection of a signal resulting from the bound probes indicates the presence of the target nucleic acid in the sample.

TMA may also be performed as part of a real-time, biphasic, target capture and amplification method. In such a method, TMA can be performed by adding amplification reagent (50 μL/test) to reaction tubes containing captured target molecules and mixing in an amplification load station. The amplification reagent contains oligos and components necessary to build nucleic acids. The reaction tubes are moved to a transition incubator at 43.7° C. to increase the temperature of the liquid in the reaction tubes, which are then moved back to the amplification load station where enzyme (25 μL/test) is added. Reaction tubes are moved to the amplification incubator set at 42.7° C. and remain in the incubator for five minutes, during which the first rounds of amplification are initiated. Reaction tubes are moved back to the amplification load station where promoter reagent (25 μL/test) is added. Reaction tubes are moved back to the amplification incubator for further rounds of target amplification. The promoter reagent contains oligos and torches. The torches are complementary to the target or internal calibrator and fluoresce when bound, generating signal in real-time. The signals for the target and internal calibrator preferably have different wavelengths and can be distinguished.

### ii. Polymerase Chain Reaction

Alternatively, PCR amplification (e.g., reverse transcriptase or real-time PCR) can be used for amplification. PCR can be performed with or without prior release of the target nucleic acid from the capture complex. The PCR reaction can be performed in the same vessel (e.g., a microfuge tube) as the capture step. The PCR reaction involves thermocycling between a high temperature of about 95° C. (e.g., 90-99° C.) for dissociation and a low temperature of about 60° C. e.g., 40-75, or 50-70 or 55-64° C.) for annealing. Typically, the number of complete thermocycles is at least 10, 20, 30 or 40. PCR amplification is performed using one or more primer pairs. A primer pair used for PCR amplification includes two primers complementary to opposite strands of a target nucleic acid flanking the region desired to be sequenced. For sequencing most of a viral genome (e.g., more than 50, 75 or 99%), the primers are preferably located close to the ends of the viral genome. For amplification of related molecules (e.g., mutant forms of the same virus present in a patient sample), the primers are preferably complementary to conserved regions of the target nucleic acid likely to be present in most members of the population. PCR amplification is described in PCR Technology: Principles and Applications for DNA Amplification (ed. H.A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

3. Detection

Detection of a nucleic acid target can be performed following capture and either during (real-time) or following (end-point) amplification by using any known method. The amplification product of RNA is often in the form of DNA resulting from RT-PCR or RNA copies resulting from TMA. Amplified nucleic acids may be detected in solution phase or by concentrating them in or on a matrix and detecting labels associated with them (e.g., an intercalating agent such as ethidium bromide). Some detection methods use probes complementary to a sequence in the amplified product and detect the presence of the probe:product complex, or use a complex of probes to amplify the signal detected from amplified products (e.g., U.S. Pat. Nos. 5,424,413, 5,451,503 and 5,849,481). Other detection methods use a probe in which signal production is linked to the presence of the target sequence because a change in signal results only when the labeled probe binds to amplified product, such as in a molecular beacon, molecular torch, or hybridization switch probe (e.g., U.S. Pat. Nos. 5,118,801, 5,210,015, 5,312,728, 5,538,848, 5,541,308, 5,656,207, 5,658,737, 5,925,517, 6,150,097, 6,361,945, 6,534,274, 6,835,542, and 6,849,412; and U.S. Pub. No. 2006/0194240 A1). Such probes typically use a label (e.g., fluorophore) attached to one end of the probe and an interacting compound (e.g., quencher) attached to another location of the probe to inhibit signal production from the label when the probe is in one conformation (“closed”) that indicates it is not hybridized to amplified product, but a detectable signal is produced when the probe is hybridized to the amplified product which changes its conformation (to “open”). Detection of a signal from directly or indirectly labeled probes that specifically associate with the amplified product indicates the presence of the target nucleic acid that was amplified.

4. Sequencing

Following amplification, a target nucleic acid as well as or instead of undergoing qualitative or quantitative detection can be sequenced. Purification if desired can be performed on a silica column (e.g., a Qiagen gravity flow column). The target nucleic acid binds to the column, where it can be washed and then eluted. Alternatively, purification can be performed using a nucleic acid probe-based purification system (e.g., U.S. Pat. Nos. 6,110,678 or 8,034,554,. US 2013/0209992 or US 2009/0286249, or. WO 2012/037531 or WO 2013/116774). The amplified target DNA can also be adapted for some sequencing formats by attachment of an adapter. The amplified DNA can be tailed by Klenow-mediated addition of nucleotides (usually a homopolymer) followed by annealing to an oligonucleotide complementary to the added tail, and ligation. Depending on the sequencing platform used, special adaptors are ligated to the template before sequencing. For example, a SMRT bell adapter is ligated to the sample template for sequencing with a Pacific Biosciences' PacBio RS sequencer (see, e.g., Travers et al. Nucl. Acids Res. (2010) 38 (15): e159).

The amplified target nucleic acid is suitable for sequence analysis by a variety of techniques. The capture of target nucleic acid can be coupled to several different formats of so-called next generation and third generation sequencing methods. Such methods can sequence millions of target templates in parallel. Such methods are particularly useful when the target nucleic acid is a heterogeneous mixture of variants. Among the many advantages, sequencing variants in parallel provides a profile of drug resistant mutations in the sample, even drug mutations present in relatively minor proportions within the sample.

Some next generation sequence methods amplify by emulsion PCR. A target nucleic acid immobilized to beads via a target capture oligomer provides a suitable starting material for emulsion PCR. The beads are mixed with PCR reagents and emulsion oil to create individual micro reactors containing single beads (Margulies et al., Nature 437, 376-80 (2005)). The emulsion is then broken and the individual beads with amplified DNA are sequenced. The sequencing can be pyrosequencing performed for example using a Roche 454 GS FLX sequencer (454 Life Sciences, Branford, Conn. 06405). Alternatively, sequencing can be ligation/detection performed for example using an ABI SOLiD Sequencing System (Life Technologies, Carlsbad, Calif. 92008). In another variation, target nucleic acids are eluted from beads having target capture oligomers and are immobilized in different locations on an array (e.g., the HiScanSQ (Illumina, San Diego, Calif. 92121)). The target nucleic acids are amplified by bridge amplification and sequenced by template directed incorporation of labeled nucleotides, in an array format (Illumina). In another approach, target nucleic acids are eluted from the target capture oligomer and single molecules are analyzed by detecting in real-time the incorporation nucleotides by a polymerase (single molecule real time sequencing or SMRT sequencing). The nucleotides can be labeled nucleotides that release a signal when incorporated (e.g., Pacific Biosciences, Eid et al., Sciences 323 pp. 133-138 (2009) or unlabeled nucleotides, wherein the system measures a chemical change on incorporation (e.g., Ion Torrent Personal Genome Machine (Life Technologies)).

Although captured target nucleic acids can be sequenced by any technique, third generation, next generation or massively parallel methods offer considerable advantages over Sanger and Maxam Gilbert sequencing. Several groups have described an ultrahigh-throughput DNA sequencing procedure (see. e.g., Cheeseman, U.S. Pat. No. 5,302,509, Metzker et al., Nucleic Acids Res. 22: 4259 (1994)). The pyrosequencing approach that employs four natural nucleotides (comprising a base of adenine (A), cytosine (C), guanine (G), or thymine (T)) and several other enzymes for sequencing DNA by synthesis is now widely used for mutation detection (Ronaghi, Science 281, 363 (1998); Binladin et al., PLoS ONE, issue 2, e197 (February 2007); Rehman et al., American Journal of Human Genetics, 86, 378 (March 2010); Lind et al., Next Generation Sequencing: The solution for high-resolution, unambiguous human leukocyte antigen typing, Hum. Immunol. (2010), doi 10.1016/jhumimm.2010.06.016 (in press); Shafer et al., J Infect Dis. 1;199(5):610 (2009)). In this approach, the detection is based on the pyrophosphate (PPi) released during the DNA polymerase reaction, the quantitative conversion of pyrophosphate to adenosine triphosphate (ATP) by sulfurylase, and the subsequent production of visible light by firefly luciferase. More recent work performs DNA sequencing by a synthesis method mostly focused on a photocleavable chemical moiety that is linked to a fluorescent dye to cap the 3′-OH group of deoxynucleoside triphosphates (dNTPs) (Welch et al. Nucleosides and Nucleotides 18, 197 (1999) & European Journal, 5:951-960 (1999); Xu et al., U.S. Pat. No. 7,777,013; Williams et al., U.S. Pat. No. 7,645,596; Kao et al, U.S. Pat. No. 6,399,335; Nelson et al., U.S. Pat. Nos. 7,052,839 & 7,033,762; Kumar et al., U.S. Pat. No. 7,041,812; Sood et al, US Pat. App. No. 2004-0152119; Eid et al., Science 323, 133 (2009)). In sequencing-by-synthesis methodology, DNA sequences are being deduced by measuring pyrophosphate release on testing DNA/polymerase complexes with each deoxyribonucleotide triphosphate (dNTP) separately and sequentially. See Ronaghi et al., Science 281: 363 365 (1998); Hyman, Anal. Biochem. 174, 423 (1988); Harris, U.S. Pat. No. 7,767,400.

B. Other Targets

Antibodies, proteins, particles and other targets can be detected by formats such as immunoprecipitation, Western blotting, ELISA, radioimmunoassay, competitive and immunometric assays. See Harlow & Lane, Antibodies: A Laboratory Manual (CSHP NY, 1988); U.S. Pat. Nos. 3,791,932; 3,839,153; 3,850,752; 3,879,262;4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876. Sandwich assays are a preferred format (see U.S. Pat. Nos. 4,376,110, 4,486,530, 5,914,241, and 5,965,375).

Competitive assays can also be used. In some methods, target antigen in a sample competes with exogenously supplied labeled target antigen for binding to an antibody detection reagent. The amount of labeled target antigen bound to the antibody is inversely proportional to the amount of target antigen in the sample. The antibody can be immobilized to facilitate separation of the bound complex from the sample prior to detection.

Lateral flow devices can also be used for detecting a target. Fluid is applied to a test strip that has been treated with a sample in which a target may be present. Labelled binding molecules pass through the strip and can be captured as they pass into a specific zone containing the sample with the target.

### VI. Sensitivity

The present methods can provide a high sensitivity of detection of an a target from a sample aliquot containing blood cells, such as whole blood, white blood cells, red blood cells, or other preparation of red blood cells. For pathogen-derived RNA targets, sensitivity can be expressed as a minimum number of pathogenic RNA copies present in a volume of whole blood. The volume of whole blood can be that contacted with lysis reagent directly, or can be that used to prepare a blood fraction, such as pelleted red cells, which are in turn contacted with the lysis reagent. Preferably the methods detect the presence of pathogenic RNA in whole blood with a sensitivity of about 2×10³ copies of ribosomal RNA/mL (equivalent to one parasite/1 mL) of whole blood or better, 2×10³ copies/5 mL of whole blood or better, 2×10³ copies/10 mL of whole blood or better, 2×10³ copies/50 mL of whole blood or better, or 2×10³ copies/100 mL of whole blood or better. Preferably the methods detect the presence of pathogenic RNA in whole blood with a sensitivity of about 8×10³ copies of ribosomal RNA/mL (equivalent to four parasites/1 mL) of whole blood or better, 8×10³ copies/5 mL of whole blood or better, 8×10³ copies/10 mL of whole blood or better, 8×10³ copies/50 mL of whole blood or better, or 8×10³ copies/100 mL of whole blood or better. Preferably the methods detect the presence of pathogenic RNA in whole blood with a sensitivity of about 24×10³ copies of ribosomal RNA/mL (equivalent to 12 parasites/1 mL) of whole blood or better, 24×10³ copies/5 mL of whole blood or better, 24×10³ copies/10 mL of whole blood or better, 24×10³ copies/50 mL of whole blood or better, or 24×10³ copies/100 mL of whole blood or better.

### VII. Work Flow

Individually lysed aliquots from a plurality of blood samples are pooled. Different blood cell samples are typically from different subjects, often from different humans. An exemplary volume for individual blood samples is about 200-500 ml. Sample aliquots for pooling typically represent a small proportion of a blood cell sample such as 1 μL to 2 ml or 100 μL to 1 ml or less than 1% or less than 0.1% of the volume of the blood cell samples. Aliquots can be pooled after individual lysis from 2, 4, 8, 16, 24, or 200 samples (among other numbers). In some methods, lysed aliquots from at least four samples are pooled. In some methods, lysed aliquots from 2-200, 4-24, 14-16, or 4-20 samples are pooled. After lysis individual lysed sample aliquots can be pooled in whole or in part. Pooling in whole means that the complete volumes of each lysed sample aliquot are pooled. Pooling means that a specimen (i.e., fraction) of each lysed sample aliquot is pooled to form a pooled lysate. Analyses can then be performed to detect targets that may be present in blood cell samples. The analyses can look only for targets of pathogens that infect red blood cells, such as Babesia and Plasmodium species, or can also look for targets of any pathogens likely to be present in plasma or serum, such as HIV-1 and -2, hepatatis B, hepatitis C, West Nile, Dengue treponema pallidum, and Trypanosoma cruzi, Plasmodium species and Anaplasma, or can look for therapeutic targets. Such targets can be analyzed separately or together, such as by multiplex amplification and detection on a microarray.

The sensitivity of the method arises in part from performing lysis of blood cells before one or more samplings in which only a fraction of an original sample is passed on to a subsequent analyses. Such sampling may arise from using only a fraction of each lysed sample aliquot for pooling, or using only a fraction of the pooled lysate for analysis or other dilution. The fractionation performed by any or all samplings performed between lysis and detection can be less than or equal to 0.5 (i.e., 50%), 0. 25 (25%), 0.2 (20%), 0.125 (12.5%), 0.1 (10%), 0.0625 (6.25%), 0.05 (5%), 0.0416 (4.16%)or 0.01 (1%) of the initial volume of the aliquot that is ultimately analyzed. Because the number of blood cells containing a target may be the same order or less than the inverse of the sampling fraction, there is a high possiblity that for sampling without lysis, no target containing cells will be passed on to the subsequent analysis from a sample aliquot. For example, if a sample has 2 infected cells per ml, and 0.1 ml of the sample is used for analysis, on average only ⅕ samples used for analysis will contain a particle to allow a positive result. However, if lysis is performed before sampling to release target molecules from the blood cells, the situation is different. If each blood cell gives rise to 1000 different target molecules on lysis, the number of target molecules greatly exceeds the inverse of the fraction sampled, and there is a high probability of detecting the target.

If the target is a pathogen-derived target that is not desired in the blood cell samples, and if the pooled lysates are negative for the tested target, the blood cell samples giving rise to the pool can be used (e.g., for blood transfusions and the like or banked for such use). Conversely, if pooled lysates are found to be positive for an undesired target, then individual aliquots from the samples previously combined can be tested to deconvolute which of the pooled samples contributed the pathogen. The blood cell sample(s) contributing the pathogen(s) can be removed from the collection of blood cell samples, and these other blood cell samples can be used (e.g., for blood transfusion and the like). If the target is one that is desired, then the testing is similar except that upon deconvolution the blood cell sample(s) containing the target are kept and can be used (e.g., harvesting a protein target for the blood cell sample(s) and/or from the donor(s)).

### EXAMPLES

### Example 1

This example compares the method of lysing infected hamster blood and then diluting the lysed sample in additional lysis buffer, to a method where the same hamster blood is first diluted with normal blood and then lysed. Condition A (Lyse-Dilute), 10 uL Babesia-infected whole hamster blood of which 59% of cells were infected was lysed in 20 uL PTM medium (250mM ammonium chloride, 14 mM sodium bicarbonate, 10 mM EDTA, and 8% lithium lauryl sulfate (LLS), with pH in the range of 7.2 to 7.6) and then serially diluted in the PTM. Condition B (Dilute-Lyse), 10 uL of the same infected hamster blood is first serially diluted with human whole blood and then lysed with PTM (1:3 (v/v)). Parasitemia of the infected hamster blood was determined by microscopy and by PCR wherein PCR results from the infected hamster blood were compared to PCR results of an in vitro transcript dilution curve. Parasitemia of the infected hamster blood was determined to be 43,130 parasites per mL (p/mL), and the dilutions for both Conditions A & B resulted in: 4,313 p/mL, 431 p/mL, 43 p/mL, 4.8 p/mL, 1.6 p/mL, 0.5 p/mL, 0.18 p/mL, 0.06 p/mL, 0.02 p/mL, and 0.01 p/mL. Lysis was done at ambient temperature for 5 minutes. Five 500 μL aliquots from each sample in the dilutions were tested for Babesia using a target capture and real-time transcription-mediated amplification (TMA) assay. Percent reactivity was determined as amplification/detection reactions having RLU signals greater than 100,000 being positive for detection of Babesia target.

Target capture was performed as generally described in U.S. Pat. No. 6,110,678. Babesia 18S rRNA was amplified and detected in each sample as generally described in U.S. Pat. Nos. 5,399,491, 5,554,516, 5,824,518 and 7,833,716. Primers used to amplify Babesia 18S rRNA in the samples were as follows:

TABLE 1

| FUNCTION | Sequence (5′-3′) |
|---|---|
| non T7 Primer | CTTGAATACTACAGCATGGAATAA |
| T7 Primer | AATTTAATACGACTCACTATAGGGAGATT |
| | CACCTCTGACAGTTAAATACGAA |
| Molecular | 5′ CAGUCCAUAAUGAAGUAGGACUG 3′ |
| Torch¹ | |
| TCO² | UAGGCCAAUACCCUACCGUCCTTTAAAAA |
| | AAAAAAAAAAAAAAAAAAAAAAAAA |

¹The molecular torch comprises Fam attached to its 5′ end and Dabcyl attached to its 3′ end. The molecular torch also comprises a linker between the underlined C and A residues (residues 7 & 8). The molecular torch also comprises 2′ O-methyl residues.
²The target capture oligomer (TCO) comprises 2′ O-methyl residues at the underlined residue positions.

The results are shown in Table 2, which compares % sample reactivity with number of particles/ml at the dilution tested. For samples diluted in whole blood, the reactivity drops to zero between a final concentration of 4.8 and 1.6 particles per ml. By contrast, when samples are diluted in PTM, 100% reactivity is obtained at least up to a final concentration of 0.18 particles per ml, which corresponds to about 200 18S ribosomal RNA copies per ml. Moreover, at 0.06 and 0.02 p/mL, the samples diluted in PTM provided 60% and 40% reactivity, respectively.

TABLE 2

| Sample | | | |
|---|---|---|---|
| Dilution | | Condition A % Reactivity | Condition B % Reactivity |
| (p/mL) | | (N = 5) | (N = 5) |
| 4,313 | p/mL | 100% | 100% |
| 431 | p/mL | 100% | 100% |
| 43 | p/mL | 100% | 100% |
| 4.8 | p/mL | 100% | 100% |
| 1.6 | p/mL | 100% | Not detectable |
| 0.5 | p/mL | 100% | Not detectable |
| 0.18 | p/mL | 100% | Not detectable |
| 0.06 | p/mL | 60% | Not detectable |
| 0.02 | p/mL | 40% | Not detectable |
| 0.01 | p/mL | Not detectable | Not detectable |

### Example 2

Twenty-four Babesia positive clinical RBC samples were obtained from the American Red Cross (ARC). These clinical samples in were each in Adsol solution (a preservation solution containing saline, adenine, dextrose and mannitol, Fenwal Laboratories, Deerfield, Ill.) and were stored at −20° C. on receipt. The samples were thawed the day of testing and were mixed thoroughly by inversion before manipulation. Each sample was to be prepared for testing individually (individual donor testing or IDT), and testing in pools using the pre-lysed pooling method.

### Individual Lysis of RBC Samples

To lyse the clinical samples, a 500 μL aliquot of a sample was admixed with 3.5mL of parasite transport medium (PTM) and then mixed by gentle inversion at least 3 times at ambient temperature and allowed to stand for 5 minutes. This lysate is the IDT lysate sample.

The PTM that was used in this example contains 250mM ammonium chloride, 14mM sodium bicarbonate, 10mM EDTA, and 8% lithium lauryl sulfate (LLS), with pH in the range of 7.2 to 7.6.

Fifteen normal negative samples from whole blood donors were obtained from Bioreclamation Inc. (Long Island, N.Y.) and were lysed by using a ratio of 1 mL aliquot of whole blood to 3 mL of PTM. An aliquot of lysed sample for each negative donor was set aside for negative controls.

### Preparation of Pools

Donor pools of 1:4, 1:8 and 1:16 were prepared using the IDT lysate for each clinical sample. The total volume of each pooled sample was 4.8 mL.
A pool of 1:4 was prepared by combining 1200 μL of the lysed clinical sample with 1200 μL for each of 3 lysed negative donors.
A pool of 1:8 was prepared by combining 600 μL of the lysed clinical sample with 600 μL for each of 7 lysed negative donors.
A pool of 1:16 was prepared by combining 300 μL of the lysed clinical sample with 300 μL for each of 15 lysed negative donors.

Once the samples were prepared, they were mixed by inversion several times before testing.

Three replicates of 500 μL for each IDT lysate, pooled, and negative control sample were tested on an automated Panther system using the Babesia TMA assay described above. A B. microti IVT panel at 100 c/mL was included as a positive control.

Results are shown in Table 3 below. The data shows that the pooled method was capable of detecting Babesia infection in all clinical samples with a pooling factor of 1:4 and in the vast majority of samples with a pooling factor up to 1:16. Sensitivity with the pooling method, followed by target capture and TMA was comparable to that of PCR of individual samples.

TABLE 3

| | Research | | 1:4 | 1:8 | 1:16 |
|---|---|---|---|---|---|
| (RBC) | PCR | IDT | (pool of 4) | (pool of 8) | (pool of 16) |
| 1 | Negative | Reactive | Reactive | NonReactive | NonReactive |
| 2 | Negative | Reactive | Reactive | Reactive | Reactive |
| 3 | Positive | Reactive | Reactive | Reactive | Reactive |
| 4 | Positive | Reactive | Reactive | Reactive | Reactive |
| 5 | Positive | Reactive | Reactive | Reactive | Reactive |
| 6 | Positive | Reactive | Reactive | Reactive | Reactive |
| 7 | Positive | Reactive | Reactive | Reactive | NonReactive |
| 8 | Positive | Reactive | Reactive | Reactive | Reactive |
| 9 | Positive | Reactive | Reactive | Reactive | Reactive |
| 10 | Positive | Reactive | Reactive | Reactive | Reactive |
| 11 | Positive | Reactive | Reactive | Reactive | Reactive |
| 12 | Positive | Reactive | Reactive | Reactive | Reactive |
| 13 | Positive | Reactive | Reactive | Reactive | Reactive |
| 14 | Positive | Reactive | Reactive | Reactive | Reactive |
| 15 | Positive | Reactive | Reactive | Reactive | Reactive |
| 16 | Positive | Reactive | Reactive | Reactive | Reactive |
| 17 | Positive | Reactive | not tested | Reactive | Reactive |
| 18 | Positive | Reactive | not tested | Reactive | Reactive |
| 19 | Positive | Reactive | not tested | Reactive | Reactive |
| 20 | Positive | Reactive | not tested | Reactive | Reactive |
| 21 | Positive | Reactive | not tested | Reactive | Reactive |
| 22 | Positive | Reactive | not tested | Reactive | Reactive |
| 23 | Positive | Reactive | not tested | Reactive | Reactive |
| 24 | Positive | Reactive | not tested | Reactive | Reactive |

### Example 3

In this example, B. microti infected hamster blood was diluted to 1 parasite per mL, pooled, and tested using nucleic acid amplification and detection for limit of detection in the pooled samples. An approximate parasitemia of B. microti infected hamster blood was calculated using microscopy. Based on the initial parasitemia estimate, a serial dilution of B. microti infected hamster blood was prepared in human whole blood. Nine separate 1 mL aliquots were made at the hamster blood dilution level estimated to have about 1 p/mL of B. microti. Each of the separate aliquots were lysed by combining the 1 ml aliquot with 3 mL of a blood cell lysis solution (aqueous solution containing about 100 mM TRIS, 25-30 mM MgCl₂, 6% (v/v) lithium lauryl sulfate, and pH between about 7.3 and 7.6). These nine lysates were tested directly and in 1:20 & 1:200 pools.

Each 1:20 pool of the nine lysates were prepared by combining 250 uL of the lysates into an additional 4.75 mL of lysis reagent. The 1:200 pool was prepared by combining 500 uL of the 1:20 pool with 4.5 mL of the lysis reagent. The nine aliquots of lysate were tested at 5 replicates using an isothermal amplification and detection assay on the automated Panther system (Hologic, Inc. San Diego, Calif.). The 1:20 pools and the 1:200 pools made from each of the nine lysates were tested at 7 replicates using isothermal amplification and detection assay on the automated Panther system. Results are shown in Table 4.

TABLE 4

| Lysate Aliquot | ~1 p/mL | 1:20 | 1:200 |
|---|---|---|---|
| Number | Aliquot Reactivity | Pool Reactivity | Pool Reactivity |
| 1 | 0/5 | 0/7 | 0/7 |
| 2 | 5/5 | 7/7 | 6/7 |
| 3 | 5/5 | 7/7 | 7/7 |
| 4 | 0/5 | 0/7 | 0/7 |
| 5 | 0/5 | 0/7 | 0/7 |
| 6 | 5/5 | 7/7 | 4/7 |
| 7 | 0/5 | 0/7 | 0/7 |
| 8 | 0/5 | 0/7 | 0/7 |
| 9 | 5/5 | 7/7 | 7/7 |

These data show that in samples containing as little as 1 p/mL, a 1:20 pool is 100% reactive. Furthermore, a 1:200 pool showed good sensitivity, though not 100% reactive. Thus, for an assay having limit of detection sensitivity requirements at 100%, the lysate pooling results shown in this examples indicate that at least a 1:20 pool meets the requirements. For an assay with less than 100% sensitivity requirements, this example indicates that a pool as dilute as 1:200 provides remarkably good results.

Although the invention has been described in detail for purposes of clarity of understanding, certain modifications may be practiced within the scope of the appended claims. All publications including accession numbers, web sites and the like, and patent documents cited in this application are hereby incorporated by reference in their entirety for all purposes to the same extent as if each were so individually denoted. To the extent difference version of a sequence, website or other reference may be present at different times, the version associated with the reference at the effective filing date is meant. The effective filing date means the earliest priority date at which the accession number at issue is disclosed. Unless otherwise apparent from the context any element, embodiment, step, feature or aspect of the invention can be performed in combination with any other.

1. A method of testing a sample aliquot containing blood cells for presence of a pathogen, comprising
(a) providing a sample aliquot from each of a plurality of samples, thereby providing a plurality of sample aliquots;
(b) separately contacting each of the sample aliquots of the [[a]] plurality of sample aliquots with a lysis reagent, whereby at least a portion of the blood cells in each of the plurality of sample aliquots lyse;
(c) pooling the lysed sample aliquots of step (b) to form a pooled lysate;
(d) testing the pooled lysate for presence of a pathogen, whereby identification of the presence of the pathogen in the pooled lysate indicates the presence of the pathogen in at least one of the sample aliquots.

2. The method of claim 1, wherein the lysis reagent comprises: (i) a buffer; (ii) lithium lauryl sulfate (LLS); and (iii) one or both of a chloride containing salt and an anti-coagulant selected from the group consisting of EDTA, EDTA-Na₂, EGTA, and combinations thereof, and wherein the reagent has a pH that is greater than 5.5.

3. The method of claim 2, wherein the buffer is sodium bicarbonate present in the reagent at a concentration of about 5 mM to about 30 mM, or at a concentration of about 14 mM, and the reagent comprises a chloride containing salt that is ammonium chloride at a concentration from about 100 mM to about 500 mM, or at a concentration of about 250 mM, and wherein the pH of the reagent is from about 7.0 to about 8.0.

4-7. (canceled)

8. The method of claim 2, wherein the buffer is a TRIS buffer present in the reagent at a concentration from about 75 mM to about 150 mM, or at a concentration of about 100 mM, the chloride containing salt is magnesium chloride present in the reagent at a concentration from about 20 mM to about 35 mM, or at a concentration of about 30 mM, and the LLS is present in the reagent at a concentration from about 4% (w/v) to about 15% (w/v).

9-12. (canceled)

13. The method of claim 1, wherein the entire volumes of each of the lysed sample aliquots are pooled, or wherein up to 25% of the volumes of each of the lysed sample aliquots are pooled.

14. (canceled)

15. (canceled)

16. The method of claim 1, wherein the plurality of sample aliquots are from 4 sample aliquots to 200 sample aliquots.

17. The method of claim 16, wherein the plurality of sample aliquots is up to 20 sample aliquots, or wherein the plurality of sample aliquots is up to 16 sample aliquots.

18. (canceled)

19. The method of claim 1, wherein if at the testing step (d) it is determined that a pathogen is present in the pooled lysate, the method further comprises the step of individually testing the sample aliquots to identify which of the samples is infected with the pathogen.

20. The method of claim 1, wherein each of the sample aliquots is selected from the group consisting of whole blood, a sample aliquot comprising red blood cells, and a sample aliquot comprising white blood cells.

21-23. (canceled)

24. The method of claim 1, wherein each sample aliquot is contacted with the lysis reagent at a volume ratio of sample aliquot to lysis reagent that is from about 1:2 to about 1:10 (v/v).

25-27. (canceled)

28. The method of claim 1, wherein the testing step (d) tests for presence of a pathogen-derived target released from the blood cells by the reagent, wherein the pathogen-derived target is derived from a pathogen selected from the group consisting of hepatitis viruses, human immunodeficiency viruses, dengue viruses, west nile viruses, flaviviruses, zika virus, and parasitic organisms.

29. The method of claim 28, wherein the pathogen-derived target is a nucleic acid target.

30. (canceled)

31. The method of claim 28, wherein the pathogen is a parasitic organism selected from the group consisting of: parasites from the genus Babesia, parasites from the genus Plasmodium, parasites from the genus Trypanosoma, parasites from the genus Leishmania, parasites from the genus Anaplasma, parasites from the genus Toxoplasma, Babesia microti, Babesia divergens, Babesia duncani, Plasmodium falciparum, Plasmodium malariae, Plasmodium ovale, Plasmodium vivax, and Plasmodium knowlesi.

32. The method of claim 29, wherein the pathogen-derived target is an RNA target.

33-37. (canceled)

38. The method of claim 29, wherein the testing step (d) comprises contacting the pooled lysate with a capture probe and an immobilized probe, the capture probe having a first segment complementary to the nucleic acid target, and a second segment complementary to the immobilized probe, wherein the nucleic acid target binds to the capture probe, and wherein the bound capture probe binds to the immobilized probe.

39. (canceled)

40. The method of claim 29, further comprising performing a transcription mediated amplification of the nucleic acid target and detecting the resulting amplification product with a detection probe.

41. The method of claim 1, wherein the testing step (d) comprises performing a nucleic acid amplification and detection reaction to detect a pathogen-derived target in the pooled lysate.

42. The method of claim 1, wherein the method is performed without a centrifugation step.

43-87. (canceled)

88. A method of separating a target from a plurality of sample aliquots, comprising the steps of:
(a) providing a sample aliquot from each of a plurality of whole blood samples, thereby providing a plurality of sample aliquots;
(b) separately performing a lysing reaction on each of the sample aliquots, wherein the lysing step comprises contacting each of the sample aliquots with a lysis reagent comprising
(i) a buffer,
(ii) lithium lauryl sulfate (LLS), and
(iii) at least one of a chloride containing salt and an anticoagulant selected from the group consisting of: EDTA, EDTA-Na₂, EGTA, and combinations thereof;
wherein the reagent has a pH that is greater than 5.5, and whereby at least a portion of the blood cells in each of the sample aliquots lyse;
(c) combining the lysed sample aliquots into a single pooled sample to form a pooled lysate; and
(d) separating a target from the pooled lysate.

89-93. (canceled)

94. The method of claim 88, wherein the solid support is a magnetic bead solid support, or wherein the solid support is a silica solid support.

95-133. (canceled)
